(19) United States
(12) Patent Application Publication
(10) Pub. No.: US 2005/0148521 A1
(43) Pub. Date: Jul. 7, 2005

(54) Anti-cancer combination and use thereof

(75) Inventors: Shmuel A. Ben-Sasson, Jerusalem (IL); Lilia Tsirulnikov, Jerusalem (IL); Vladimir Vainstein, Jerusalem (IL)

Correspondence Address: David S. Resnick;NIXON PEABODY LLP, 100 Summer Street, Boston, MA, 02110-2131, US

(73) Assignees: Yissum Research Development Company of the Hebrew University, Jerusalem (IL); The Children's Medical Center Corporation, Boston, MA (US)

(21) Appl. No.: 10/898,721

(22) Filed: Jul. 23, 2004

Related U.S. Application Data
(63) Continuation-in-part of application No. PCT/US02/41767, filed on Dec. 31, 2002.
(60) Provisional application No. 60/351,946, filed on Jan. 24, 2002.

(51) Int. Cl. A61K 31/704; A61K 31/4745; A61K 31/405; A61K 31/675; A61K 31/525; A61K 31/337; A61K 31/522; A61K 31/195; A61K 31/12

(52) U.S. Cl. 514/34; 514/89; 514/102; 514/109; 514/263.31; 514/483; 514/567; 514/420; 514/251; 514/449; 514/690; 514/532

(57) ABSTRACT

The present invention relates to the surprising discovery that the combination of several agents, each well known for its established role in treating cancer, inflammation, hemostasis, bone resorption or serving as a solubilizing vehicle, results in a synergistic anti-cancer composition. Furthermore, the combination of at least three agents allows the cytotoxic agent, such as cyclophosphamide, to be used at a lower dosage than when administered alone. One predicted consequence of this treatment, therefore, is a highly desirable reduction in toxic side effects due to the cytotoxic agent.

### CROSS-REFERENCE

This application is a continuation-in-part and claims benefit under 35 U.S.C. § 120 of co-pending International Application No. PCT/U.S. 2002/041767 filed on Dec. 31, 2002, designating the United States, which claims benefit under 35 U.S.C. § 119(e) to U.S. Provisional Application No. 60/351,946 filed Jan. 24, 2002.

### BACKGROUND OF THE INVENTION

The treatment of cancer has thus far proved problematic. While “cancers” share many characteristics, each particular cancer has its own specific characteristics. Genetics and environmental factors have a complex interplay in the severity and prognosis of treatment. Thus, treatment must be carefully tailored.

Although cancer chemotherapy has advanced dramatically in recent years, treating cancers with a single agent has had limited success. First, any single agent may only target a subset of the total population of malignant cells present, leaving a subpopulation of cancerous cells to continue growing. Second, cells develop resistance upon prolonged exposure to a drug. Combination therapies, which employ two or more agents with differing mechanisms of action and differing toxicities, have been useful for circumventing drug resistance and increasing the target cell population. In addition, certain combinations of agents may be synergistic: their combined effect is larger than that predicted based on their individual activities. Thus, combining different agents can be a powerful strategy for treating cancer. However, combination therapies are a hit or miss proposition. In many cases, cross effects and treatment load can result in lower effectiveness for the combination than either treatment alone. Multidrug resistance can also be a problem.

Cytotoxic agents such as cyclophosphamide have been used to treat cancers. The most striking difference between malignant and healthy cells is the capacity of cancer cells for unrestricted proliferation. This difference is exploited by many cytotoxic agents, which typically disrupt cell proliferation by interfering with the synthesis or integrity of DNA. Examples of classes of cytotoxic agents which function in this manner include alkylating agents (e.g. nitrogen mustards such as cyclophosphamide), antimetabolites (e.g. purine and pyrimidine analogues), and platinum coordination complexes.

One problem with cytotoxic agents which function by disrupting cell division is that they don't discriminate between healthy and malignant cells: any dividing cell is a potential target for their action. Thus, cell populations which normally exhibit high levels of proliferation (such as bone marrow) are targeted, leading to the toxic side effects commonly associated with cancer treatments.

Steroidal anti-inflammatory agents, known as the glucocorticoids, and non-steroidal anti-inflammatory drugs, known as NSAIDs, are among the most frequently prescribed drugs and are typically used to treat diseases resulting from undesirable immune reactions. Some examples of diseases treated with glucocorticoids or NSAIDs include rheumatic disorders, allergy, asthma, and transplantation rejection. Their anti-inflammatory effect is mediated by an inhibition of prostaglandin production and decreased accumulation of macrophages and leukocytes at sites of inflammation.

Despite their utility in treating a wide variety of diseases, glucocorticoids and NSAIDs are not traditionally used to treat cancer. Anti-inflammatory agents are usually considered to be counter-productive for the treatment of cancer, as the immune system may help the body fight certain cancers. (An exception to this is the use of glucocorticoids to treat malignancies of the immune system such as lymphoma.) In fact, the combined use of an anti-inflammatory agent and a cytotoxic anti-cancer agent, e.g. dexamethasone and cyclophosphamide, has been suggested to be avoided as it is believed that glucocorticoids reduce the activity of cyclophosphamide (Nursing 98 Drug Handbook, p. 891, Springhouse Co., PA, 1998).

Some NSAIDs acts via the inhibition of the enzymes cyclooxygenase 1 and/or 2 (=COX1 and/or COX2). There were suggestions and hopes that anti-inflammatory agents, which are selectively COX2 inhibitors, will have a beneficial effect on cancer (Ziegler J. J. Natl. Cancer Inst. 91: 1186 (1999)). However, a recent study shows that this is not the case (Dolora P. et al. Scand. J. Gastroenterol. 34: 1168 (1999)). Our own studies with tumor-bearing mice also show no significant anti-cancer effect of the COX2 inhibitor Rofecoxib (Vioxx), when given by itself.

Inhibitors of pro-angiogenic growth factors are agents used to inhibit the signaling of known pro-angiogenic factors like VEGF or FGF. Such agents can act extracellularly, by the inhibition of the interaction of an angiogenic factor with its receptor or can act intracellularly via the inhibition of the protein-kinase activity of the corresponding receptors. These agents include, for example, anti-VEGF or anti-VEGF-Receptor antibodies or inhibitors of the protein-kinase domain of VEGF-R, FGF-R or PDGF-R. Currently, these agents by themselves failed to demonstrate sufficient efficacy in the treatment of cancer.

With only a few exceptions, no single drug or drug combination is curative for most cancers. Thus, new drugs or combinations that can delay the growth of life-threatening tumors and/or improve quality of life by further reducing tumor load are very important.

Bisphosphonates are chemical analogs of pyrophosphate that are resistant to hydrolysis by pyrophosphatase and have become the most commonly used drug for the treatment of hypercalcemia. Bisphosphonates adsorb to the surface of crystalline hydroxyapatite and inhibit calcium release from bone. Therefore, bisphosphonates are being used as an adjuvant therapy for the treatment of bone metastases (common in breast cancer and prostate cancer) because they are potent inhibitors of osteoclastic bone resorption with proven efficacy in reducing tumor associated skeletal complications (Gralow J R. Curr. Oncol. Rep. 3: 506 (2001)).

Quinones are known for their ability to induce oxidative stress through redox cycling, hereby referred to as redox quinones (for a review see Powis G., Free Radic. Biol. Med. 6: 63-101 (1989)). Of special therapeutic value are pharmaceutically acceptable redox quinones such as Vitamin Ks required for the bioactivation of proteins involved in hemostasis. Vitamin K₃ in particular is well known for its redox efficacy. Vitamin K₃, also known as menadione or 2-methyl-1,4-naphthalenedione, includes a hydrophobic form and a water soluble, sodium bisulfit form. Vitamin K₃ serves as a prothrombogenic agent, mainly in supplement of veterinary diet. Studies that specifically examined whether Vitamin K₃ can be beneficial for cancer chemotherapy failed to show any activity of Vitamin K₃ as an anti-cancer agent (see Tetef M. et al. J. Cancer Res. Clin. Oncol. 121: 103-6 (1995)).

Benzyl benzoate is an example of an ester of benzoic acid that is being used as a vehicle in the formulation of a variety of drugs.

### SUMMARY OF THE INVENTION

The present invention relates to the surprising discovery that the combination of several agents, each well known for its established role in treating cancer, inflammation, hemostasis, bone resorption or serving as a solubilizing vehicle, results in a synergistic anti-cancer composition. Furthermore, the combination of at least three agents allows the cytotoxic agent, such as cyclophosphamide, to be used at a lower dosage than when administered alone. One predicted consequence of this treatment, therefore, is a highly desirable reduction in toxic side effects due to the cytotoxic agent.

Accordingly, the present invention relates to an antineoplastic/antiangiogenic combination of at least three agents, and to a method for treating cancer, macular degeneration or obesity comprising administering each agent. The invention more particularly provides a composition comprising a cytotoxic agent, preferably cyclophosphamide, an anti-inflammatory agent, preferably a COX 1-2 inhibitor such as diclofenac and indomethacin, an ester of benzoic acid, preferably Benzyl benzoate, and a pharmaceutically acceptable carrier. In certain embodiments, the combination further includes a bisphosphonate, preferably pamidronate or alendronate. In other embodiments, the combination further includes a matrix metalloproteinase (MMP) inhibitor. In additional embodiments, the combination further includes a redox quinone, preferably Vitamin K₃.

A preferred composition comprises benzyl benzoate, diclofenac or indomethacin, Vitamin K₃ and cyclophosphamide or ifosfamide.

As used herein, the phrase “steroidal anti-inflammatory agent” means a glucocorticoid, including, for example, dexamethasone, betamethasone, triamcinolone, 6a-methylprenisolone, prednisolone, prednisone, hydrocortisone, cortisone, and fludrocortisone. Preferred steroidal anti-inflammatory agents include dexamethasone, betamethasone, triamcinolone, 6a-methylprenisolone, prednisone, and prednisolone. Dexamethasone and prednisone are most preferred steroidal anti-inflammatory agents.

As used herein, the phrase “NSAIDs” typically means non-steroidal drugs that are cyclooxygenase inhibitors (COX1 and/or COX2), including for example, Salicylic acid derivatives such as aspirin, sodium salicilate, choline magnesium trisalicylate, salsalate, diflunisal, salicylsalicylic acid, sulfasalazine, olsalazine. Para-aminophenol derivatives such as acetaminophen. Indole and indene acetic acids such as indomethacin, sulindac, etodolac. Heteroaryl acetic acids such as tolmetin, diclofenac, ketorolac. Arylpropionic acids such as ibuprofen, naproxen, flurbiprofen, ketoprofen, fenoprofen, oxaprozin. Anthranilic acids (fenamates) such as mefenamic acid, meclofenamic acid. Enolic acids such as oxicams (piroxicam, tenoxicam), pyrazolidinediones (phenylbutazone, oxyphenthatrazone). Alkanones such as nabumetone. Preferred NSAIDs include COX2 inhibitors such as celecoxib, rofecoxib, valdecoxcib, etoricoxib and COX-189 and COX 1-2 inhibitors such as diclofenac and indomethacin. COX 1-2 inhibitors are preferred. Combinations including COX 1-2 inhibitors need not include a bisphosphonate.

As used herein, the phrase “cytotoxic agent” means an agent used to treat abnormal and uncontrolled progressive cellular growth. Preferred cytotoxic agents include, for example, cyclophosphamide, ifosfamide, cytarabine, 6-mercaptopurine, 6-thioguanine, vincristine, doxorubicin, and daunorubicin, chlorambucil, carmustine, vinblastine, methotrexate, and paclitaxel. More preferred cytotoxic agents include cyclophosphamide, ifosfamide, cytarabine, 6-mercaptopurine, 6-thioguanine, vincristine, mitoxantrone, doxorubicin, and daunorubicin. Cyclophosphamide and ifosfamide are most preferred cytotoxic agents.

As used herein, the phrase “bisphosphonates” means chemical analogs of pyrophosphate that are resistant to hydrolysis by pyrophosphstase and are used for the treatment of hypercalcemia. Preferred bisphosphonates include, for example, etidronate, pamidronate, clodronate, alendronate, tiludronate, ibandronate and risedronate. More preferred bisphosphonates include pamidronate and alendronate. Bisphosphonates are preferably excluded when higher doses of COX 1-2 inhibitors are used. With diclofenac, for example, when the dose administered is around 5 mg/Kg and above bisphosphorates are preferably excluded.

As used herein, the phrase “ester of benzoic acid” means non-substitute aromatic or alkyl esters, like benzyl benzoate or esters where either the aromatic alcohol and/or the aromatic acid (the benzoate) are substituted. Examples of alkyl esters of benzoic acid include substituted and non-substituted benzoate. Examples of alkyl esters of substituted benzoate are ethyl ₃-hydroxybenzoate or n-butyl p-aminobenzoate (these examples are just for illustration because of numerous possibilities). Benzyl benzoate is an example of such agent used as a vehicle in the formulation of a variety of drugs. Preferred ester of benzoic acid includes, for example, benzyl benzoate. The present invention includes an oral formulation of benzyl benzoate. Due to the hydrophobic nature of the aromatic ester the formulation includes pharmaceutically acceptable surface active agents such as non-ionic detergents like cremophor EL, Solutol HS15, Pluronic (BASF), poloxamers, Tween-20 and Tween-80 or ionic detergents like bile salts (e.g. sodium deoxycholate).

As used herein, the phrase “inhibitors of pro-angiogenic growth factors” means agents used to inhibit the signaling of known pro-angiogenic factors like VEGF or FGF. Such agents can act extracellularly, by the inhibition of the interaction of an angiogenic factor with its receptor or can act intracellularly via the inhibition of the protein-kinase activity of the corresponding receptors. These agents include, for example, anti-VEGF or anti-VEGF-Receptor antibodies (U.S. Pat. No. 6,416,758 and WO 01/72829) or inhibitors of the protein-kinase domain of VEGF-R, FGF-R or PDGF-R (WO 97/34876 and U.S. Pat. No. 6,462,060). Currently, these agents by themselves failed to demonstrate sufficient efficacy in the treatment of cancer.

As used herein, the phrase “redox quinones” means quinones that are capable of inducing oxidative stress through redox cycling. Such quinones can be pharmaceutically acceptable agents such as Vitamin K₃.

This oral formulation includes a combination with redox quinine, preferably Vitamin K₃. A mixture of benzyl benzoate and Vitamin K₃ may be used in the combination.

As used herein, the phase “matrix metalloproteinase (MMP) inhibitor” means any chemical compound that inhibits by at least five percent the hydrolytic activity of at least one matrix metalloproteinase enzyme that is naturally occurring in a mammal. Such compounds are also referred to as “MMP inhibitors”. Numerous matrix metalloproteinase inhibitors are known, and all are useful in the present invention.

In a further embodiment of the invention, we provide a method for treating cancer, macular degeneration and obesity comprising administering to hosts in need of treatment an effective amount of a combination of a redox quinone, a COX1-2 inhibitor, an ester of benzoic acid, a cytotoxic agent, and a pharmaceutically acceptable carrier. In certain embodiments, a bisphosphonate and/or MMP inhibitor and/or inhibitor of pro-angiogenic growth factor is added. The host includes a human or domestic animal, e.g. a cat or dog.

In yet a further embodiment of the present invention, we provide a formulation for treating cancer with the above drug combination. Said formulation includes a controlled-release device where one or several of the drugs are being released in a delayed fashion. Such formulation can be in the form of a tablet (or a pill) which releases different doses of drugs in different time intervals after being taken orally.

A preferred method embraces the treatment of solid tumors and leukemias, including lung cancer, colorectal cancer, breast cancer, prostate cancer, and melanoma.

The present invention further includes a method of treatment comprising oral administration of a drug-combination for treating cancer, macular degeneration and obesity where the daily composition is not identical, e.g. a two day or a three day cycle with a non-identical daily composition. In a preferred embodiment, oral administration is a weekly cycle. For example: Sunday and Wednesday cyclophosphamide, benzyl benzoate, Vitamin K₃ and diclofenac; while benzyl benzoate and Vitamin K₃-only during the rest of the week. Alternatively: Sunday and Wednesday cyclophosphamide, Vitamin K₃ and benzyl benzoate; Monday and Thursday diclofenac, Vitamin K₃ and benzyl benzoate; Tuesday and Friday benzyl benzoate and Vitamin K₃ only; Saturday placebo (in order to keep with patient compliance). The present invention also includes a kit having components of the combination and directions for their administration. The Vitamin K₃ may be formulated with benzyl benzoate.

### BRIEF DESCRIPTION OF THE DRAWINGS

FIG. 1 illustrates the results of Experiment 1. In each group, the treatments are as follows:
Group 1. Vehicle only (Control)
Group 2. Benzyl benzoate 550 mg/kg (A) and Pamidronate 10 mg/kg (B)
Group 3. B and Rofecoxib 10 mg/kg (C)
Group 4. A and C

FIG. 2 illustrates the results of Experiment 2. In each group, the treatments are as follows:
Group 1. Benzyl benzoate+Pamidronate+Rofecoxib (ABC)
Group 2. Vehicle only (Control).

FIG. 3 illustrates the results of Experiment 3. In each group, the treatments are as follows:
Group 1. Vehicle only (Control)
Group 2. Cyclophosphamide 50 mg/kg (CTX 50)
Group 3. ABC
Group 4. ABC+CTX 50

FIG. 4 illustrates the results of Experiment 4. In each group, the treatments are as follows:
Group 1: Vehicle only (Control)
Group 2: CTX50
Group 3: CTX50+ABC
Group 4: (CTX50+BC)×2 per week+1/6A×6 per week (A all week)

FIG. 5 illustrates the results of Experiment 5. In each group, the treatments are as follows:
Group 1: Vehicle only (Control)
Group 2: (BC+CTX50)×2 per week; A all week (Sun-Fri)
Group 3: (BC+Cyclophosphamide 100 mg/kg (CTX100)) once a week; A all week (Sun-Fri)
Group 4: BC Wed; CTX100 Sun; A all week (Sun-Fri)

FIG. 6 illustrates the results of Experiment 6. In each group, the treatments are as follows:
Group 1: Vehicle only (Control)
Group 2: BC Wed; CTX100 Sun; A all week
Group 3: twice the dose of C (2C) Wed; CTX100 Sun; A all week
Group 4: B+Diclofenac 25 mg/kg (D) Wed; CTX100 Sun; A all week
Group 5: twice the dose of D (2D) Wed; CTX100 Sun; A all week

FIG. 7 illustrates the results of Experiment 7. In each group, the treatments are as follows:
Group 1: Vehicle only (Control)
Group 2: CTX100 Sun; Diclofenac 50 mg/kg (D50) Mon; A all week
Group 3: CTX100 Sun; D50 Mon; A all week
Group 4: CTX100 Sun; D50 Fri; A all week
Group 5: CTX20 all week (Sun-Fri); D50 Wed; A all week

FIG. 8 illustrates the results of Experiment 8. In each group, the treatments are as follows:
Group 1: Vehicle only (Control)
Group 2: (A+VK20) all week
Group 3: (CTX60+D30+VK50)M,H+(A) all week
Group 4: (CTX60+D30)M,H+(A+VK20) all week

FIG. 9 illustrates the blood level of the CEA marker of a colon cancer patient (#4). CEA leveled off following the initiation of the treatment.

FIG. 10 illustrates the blood level of the CEA marker of a colon cancer patient (#7). As shown in the figure, CEA levels raised significantly before treatment. Following treatment, CEA stabilized.

FIG. 11 illustrates that the PSA levels of a prostate cancer patient were rising in face of hormonal treatment. PSA levels started to drop after the initiation of the drug-combination treatment (arrows). Vehicle only (Control).

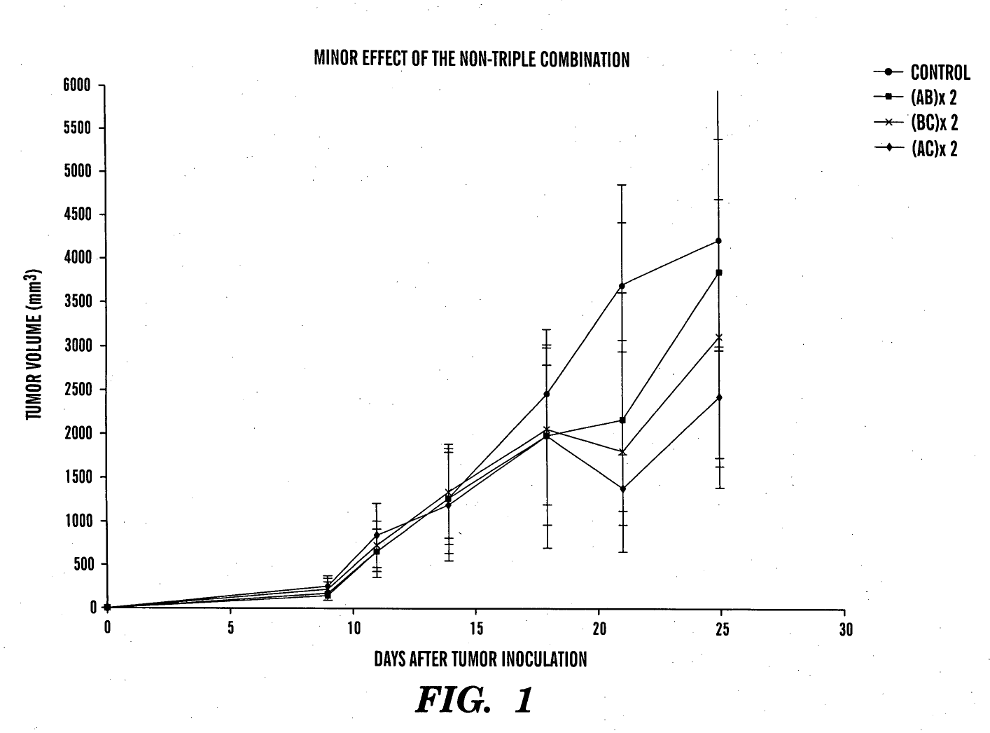

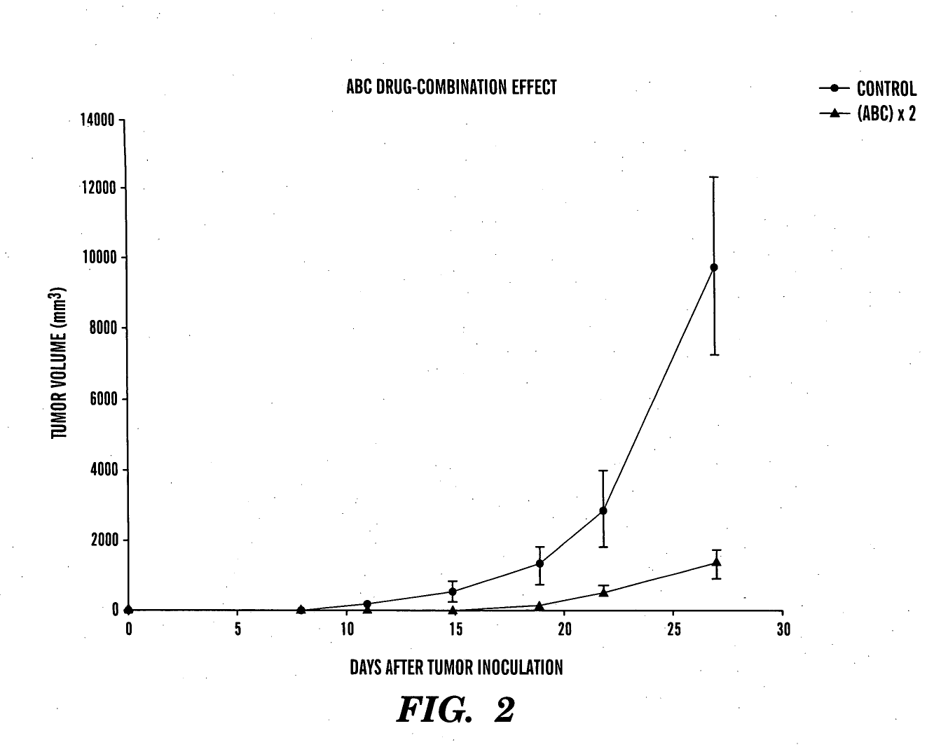

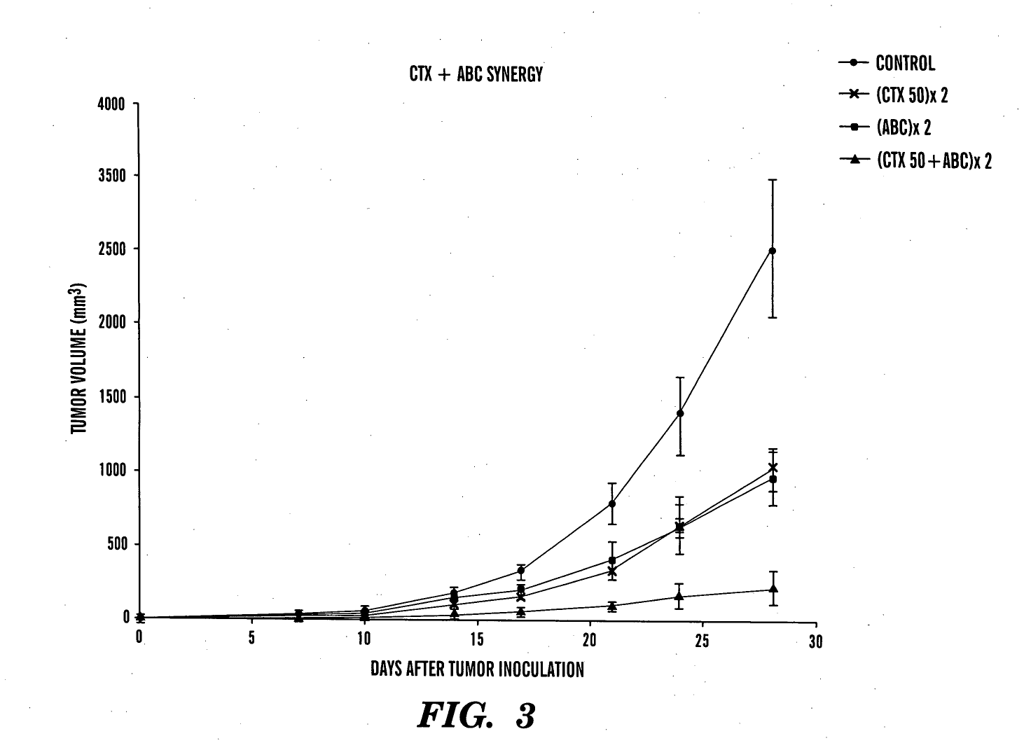

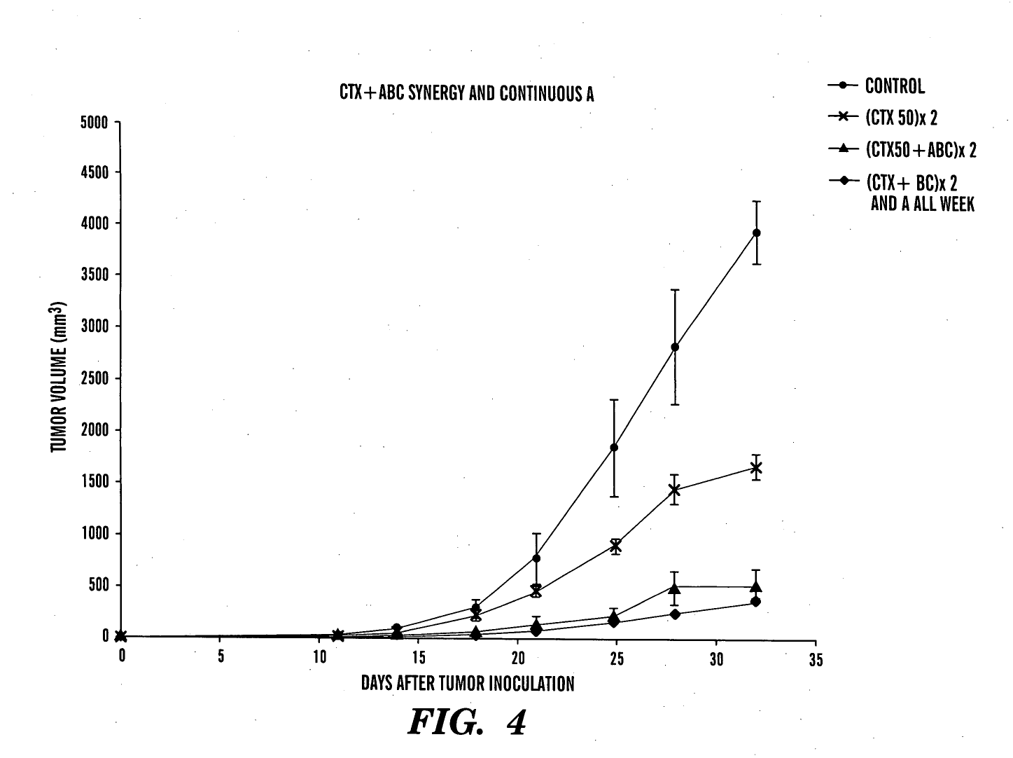

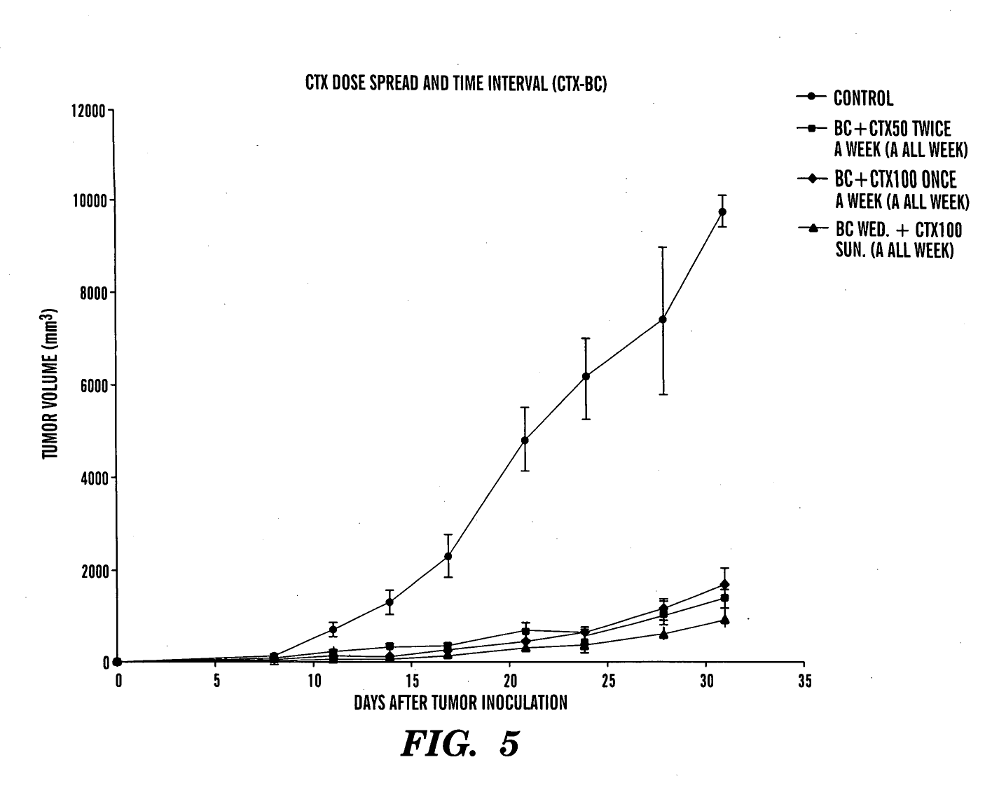

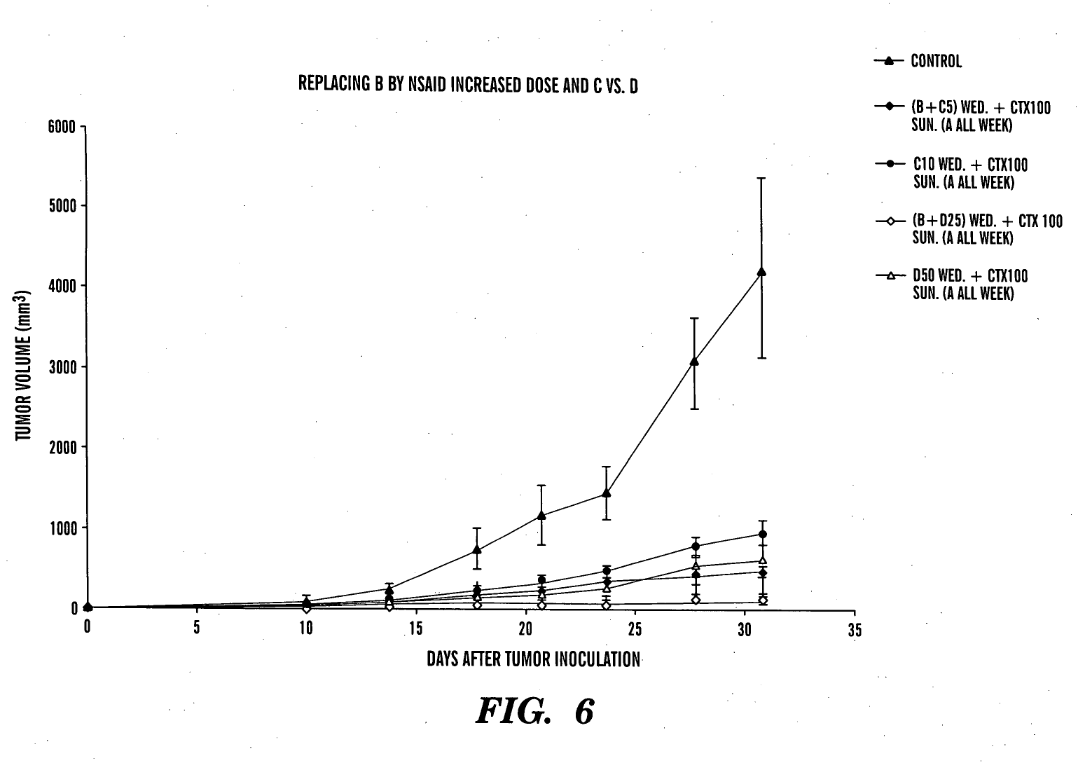

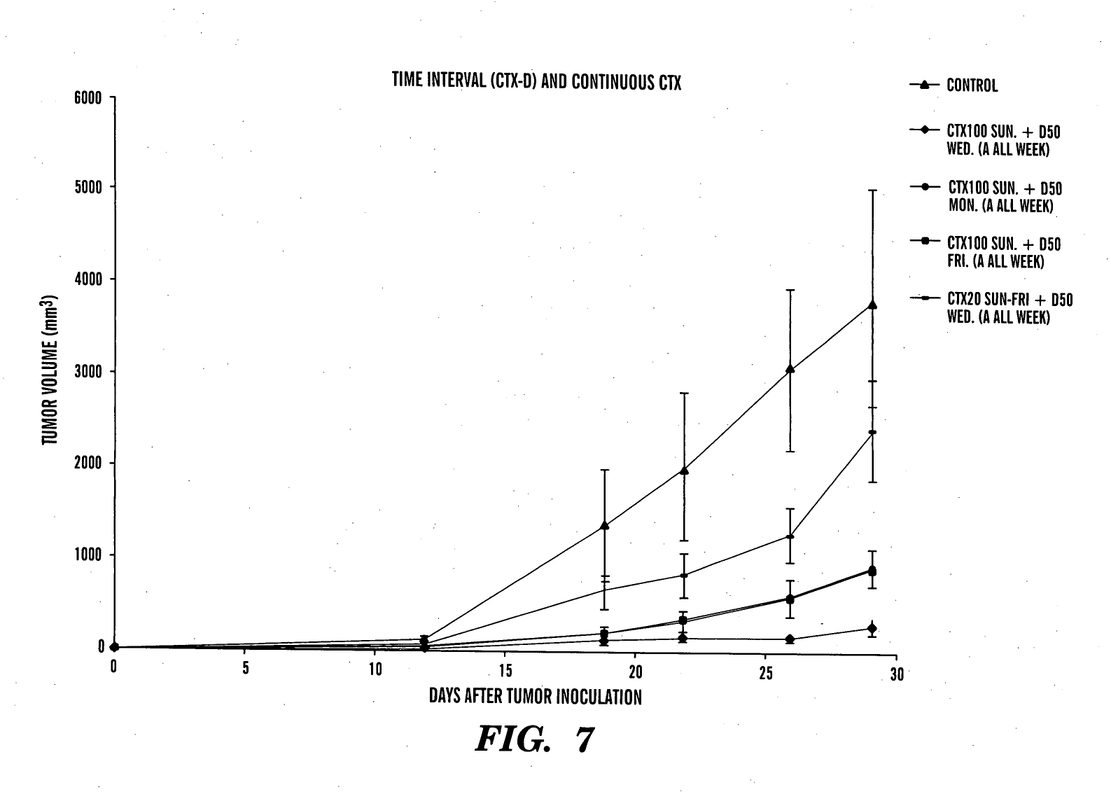

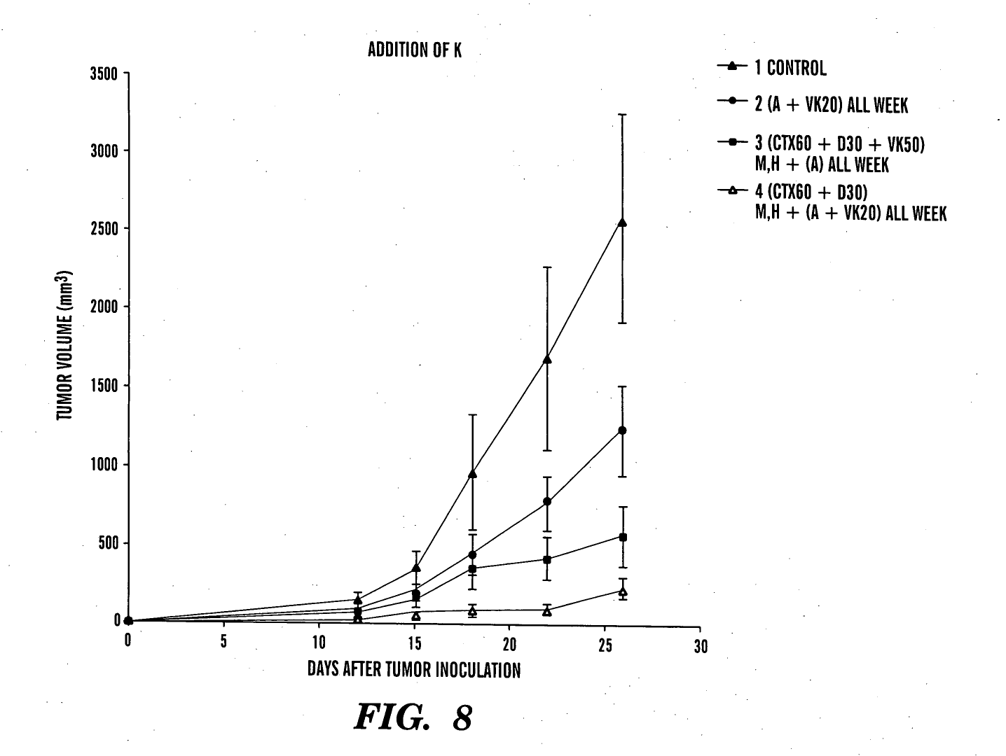

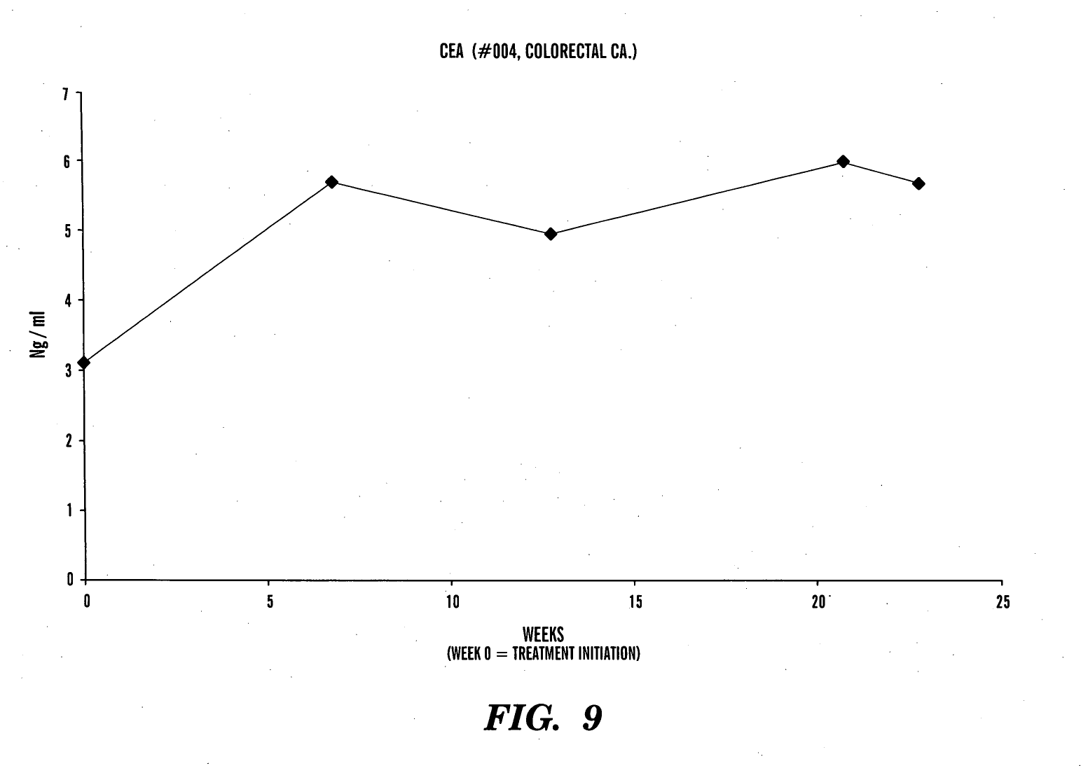

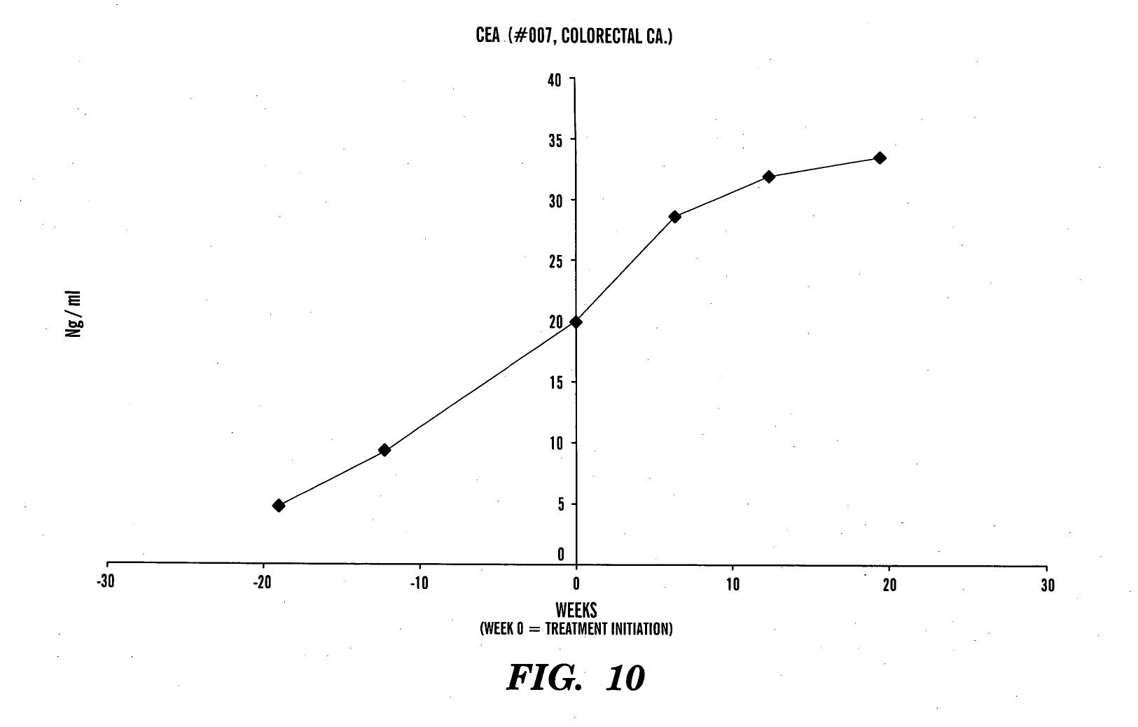

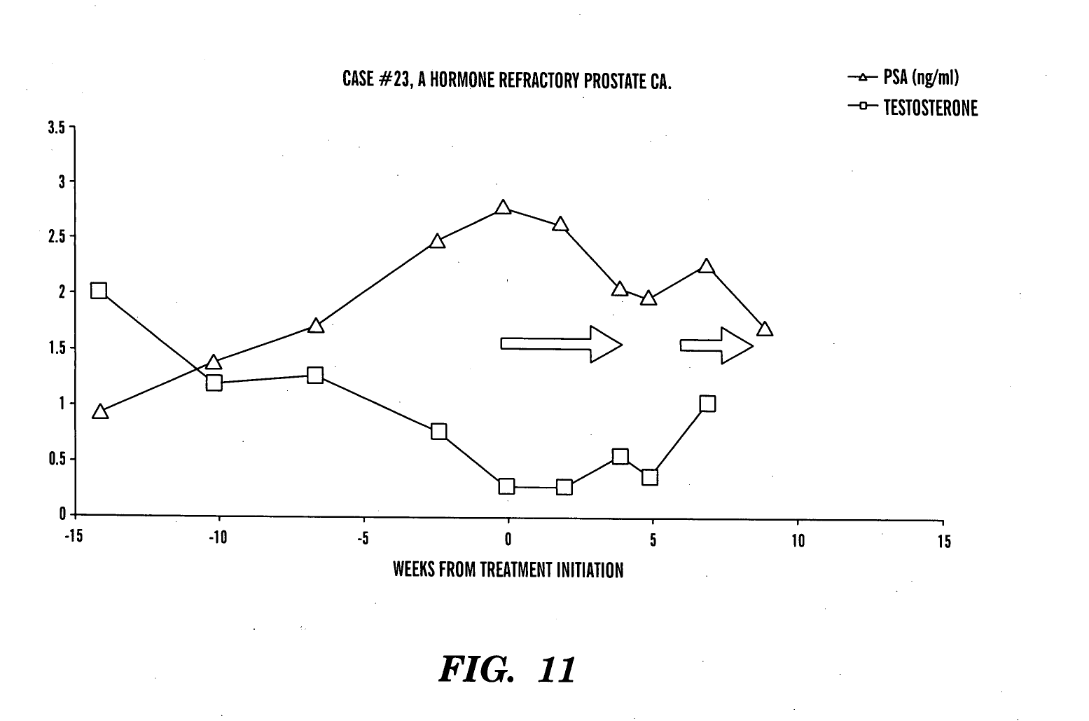

### DETAILED DESCRIPTION OF THE INVENTION

This invention provides for advantageous combination therapies for solid tumors and leukemias, macular degeneration or obesity using methods which employ administration of a NSAID agent (preferably a COX1-2 inhibitor), a cytotoxic agent, an ester of benzoic acid (preferably benzyl benzoate), redox quinone, preferably Vitamin K₃ and optionally, a bisphosphonate agent and/or MMP inhibitor and/or inhibitors of pro-angiogenic growth factors. The combination of the present invention results in a surprising reduction in tumor size.

The pharmaceutical combination or each agent individually can be administered by any means known in the art. Such modes include oral, rectal, nasal, topical (including buccal and sublingual), or parenteral (including subcutaneous, intramuscular, intravenous, and intradermal) administration, including sustained release formulations.

For ease to the patient oral administration is preferred. However, typically oral administration requires a higher dose than an intravenous administration. Thus, administration route will depend upon the situation: the skilled artisan must determine which form of administration is best in a particular case, balancing dose needed versus the number of times per month administration is necessary.

In administering the compounds one can use the normal dose of each compound individually. However, with regard to the cytotoxic agent, in order to reduce side effects, preferably one uses a lower level than used when given as a single cytotoxic agent—typically 75% or less of the individual amount, more preferably 50% or less, still more preferably 40% or less. Conversely, with regard to the NSAID, MMP inhibitor, the bisphosphonate agent, redox quinone and the ester of benzoic acid, a dose equal to or higher than the recommended dose for their corresponding conventional indications is used. Preferably, one uses a higher dose—typically 25% or more than the highest recommended dose, more preferably 50% or more, still more preferably 100% or more.

The individual compounds will be addressed in more detail below.

The first component of the combination therapy described is an anti-inflammatory agent. Non-steroidal anti-inflammatory drugs, known as the NSAIDs, are among the most frequently prescribed agents and are typically used to treat diseases resulting from undesirable immune reactions. The biochemical effects of NSAIDs are widespread and diverse, including profound effects on COX1 and/or COX2. Their anti-inflammatory effect is mediated by an inhibition of prostaglandin production and decreased accumulation of macrophages and leukocytes at sites of inflammation.

The invention can utilize a variety of NSAIDs. Because this group of agents is so vast, only one example of each class of NSAIDs is detailed here. Possible NSAIDs for the composition of the invention include but are not limited to salicylic acid derivatives (such as aspirin, Bristol-Meyers Squibb), heteroaryl acetic acids (such as diclofenac, Novartis), para-aminophenol derivatives (such as acetaminophen, McNeil Consumer), indole and indene acetic acids (such as indomethacin, Merck), aryl propionic acids (such as ibuprofen, Mylan), anthranilic acids (such as mefenamic acid, Parke-Davis), enolic acids (such as piroxicam, Teva), and alkones (such as nabumetone, SmithKline Beecham). A preferred NSAID is a COX2 inhibitor such as celecoxib (=Celebrex, Merck), rofecoxib (=Vioxx, Pfizer/Searle), valdecoxcib, etoricoxib and COX-189. A most preferred NSAID is a COX 1-2 inhibitor such as diclofenac, (Norvatis) or indomethacin (Merk).

The NSAID may be administered in any manner found appropriate by a clinician, such as those described in the Physicians' Desk Reference, 56^{th} Ed. (2002) Publisher Edward R. Barnhart, New Jersey (“PDR”). For example, when the NSAID is a COX1-2 inhibitor such as diclofenac, the dosage is 0.1-100 mg/kg; preferably 1-10 mg/kg.

The second component of the combination therapy described is a cytotoxic agent. Currently available cytotoxic drugs can be broadly divided by their mechanism of action into four groups: alkylating agents, anti-metabolites, antibiotics, and miscellaneous other activities. The choice of a particular cytotoxic agent to treat an individual with cancer is influenced by many factors, including the type of cancer, the age and general health of the patient, and issues of multidrug resistance.

The composition of the invention can utilize a variety of cytotoxic agents, including but not limited to the following agents (including possible sources): the alkylating agents cyclophosphamide (Bristol-Meyers Squibb), ifosfamide (Bristol-Meyers Squibb), chlorambucil (Glaxo Wellcome), and carmustine (Bristol-Meyers Squibb); the anti-metabolites cytarabine (Pharmacia & Upjohn), 6-mercaptopurine (Glaxo Wellcome), 6-thioguanine (Glaxo Wellcome), and methotrexate (Immunex); the antibiotics doxorubicin (Pharmacia & Upjohn), daunorubicin (NeXstar), and mitoxantrone (Immunex); and miscellaneous agents such as vincristine (Lilly), vinblastine (Lilly), and paclitaxel (Bristol-Meyers Squibb). Preferred cytotoxic agents include cyclophosphamide, ifosfamide, cytarabine, 6-mercaptopurine, 6-thioguanine, doxorubicin, daunorubicin, mitoxantrone, and vincristine. The most preferred cytotoxic agent are cyclophosphamide and ifosfamide.

The cytotoxic agent may be administered in any manner found appropriate by a clinician, such as those described for individual cytotoxic agents in the PDR. For example, when the cytotoxic agent in cyclophosphamide, the dose is preferably 0.1-50 mg/kg, most preferably 0.2-20 mg/kg.

The third component of the combination therapy described is an aliphatic (alkyl) or aromatic ester of benzoic acid. Examples of alkyl esters of benzoic acid, include substituted and non-substituted benzoate. Examples of alkyl esters of substituted benzoate are ethyl ₃-hydroxybenzoate or n-butyl p-aminobenzoate (these examples are just for illustration because of numerous possibilities). An aromatic ester of benzoic acid means non-substitute aromatic esters, like benzyl benzoate or esters where either the aromatic alcohol and/or the aromatic acid (the benzoate) are substituted. Benzyl benzoate (CAS # 120-51-4) is an example of such agent used as a vehicle in the formulation of a variety of drugs. Benzyl benzoate is a preferred ester of benzoic acid. Benzyl benzoate is available commercially from BF Goodrich Kalama, Inc. (Kalama, Wash.).

The ester of benzoic acid may be administered in any manner found appropriate by a clinician such as those described known in the drug formulation art. For example, when the ester of benzoic acid is benzyl benzoate the dosage is preferably 0.2-200 mg/kg, most preferably 1-50 mg/kg. Additionally, it is preferred that the benzyl benzoate not be administered as a pure solution but is diluted to make a suspension of up to 20% (v/v) benzyl benzoate in aqueous solution, preferably a 2%-10% suspension.

The fourth component of the combination therapy described is redox quinine, preferably Vitamin K₃. The dosage is preferably 0.1-100 mg/kg, most preferably 0.5-20 mg/kg. The Vitamin K₃ may be formulated with benzyl benzoate and administered as part of the combination therapy.

The fifth component of the combination therapy described is an inhibitor of an MMP. Examples of inhibitors of an MMP include 11,10-phenanthroline (o-phenanthroline); batarnistat also known as BB-94, [4-(N-hydroxyamino)-2R-isobutyl-3S-(thiopen ylthiomethyl)-succinyl]-L-phenylalanine-N-methylamidecarboxy-alkylamino-based compounds such as N+1-(R)-carboxy(1,3-dihydro-2H-benz[f]isoindol yl)propyl]-N′,N′-dimethyl-L-leucinamide,trifluoroacetate (J. Med Chem. 36: 4030-4039, 1993); marimastat (BB-2516); N-chlorotaurine; eicosapentaenoic acid; matlystatin-B; actinonin (3-[[1-[[2-(hydroxymethyl)-1-pyrolidinyl]carbamoyl]-octanohydroxamic acid); N-phosphonalkyl dipeptides such as N-[N-((R)-1-phosphonopropyl-(S)-leucyl]-(S)-phenylalanine-N-methylamide (J. Med. Chem. 37: 158-169, 1994); peptidylhydroxamic acids such as pNH2-Bz-Gly-Pro-D-Leu-D-Ala-NHOH (Biophys. Biochem. Res. Comm. 199: 1442-1446, 1994); Ro 7467, also known as 2+5-bromo-2,3-dihydro hydroxy-1,3-dioxo-1Hbenz[de]isoquinolin yl)methyl](hydroxy)-[phosphinyl]-N-(2-oxo azacyclotridecanyl) methylvaleramide;CT 1 166, also known as N I f N-[2-(morpholinosulphonylamino)-ethyl]-3-cyclohexyl-1 5 2-(S)-propanamidyl 1-N-4-hydroxy(R)-[3-(4-methylphenyl)propyl]-succinamide(Biochem. J. 308:167-175, 1995); bromocyclic-adenosine monophosphate;protocatechuic aldehyde (3,4-dihydroxybenzaldehyde); estramustine (estradiol bis(2-chloroethyl)carbamate). Yet another example of MMP inhibitors are doxycycline or CMT-8 (J. Periodontol. 73: 726-734, 2002).

A particular inhibitor may inhibit more than one MMP. The inhibitor may inhibit, for example, MMP-1 (interstitial collagenase), MMP-2 (72 kDcollagenase), MW-3 stromelysin), MMP-4 (telopeptidase), MMP-5 (collagenendopeptidase), NIMP-6 (acid metalloproteinase), MMP-7 (uterinemetalloproteinase), MMP-8 (neutrophil collagenase), and/or MMP-9 (92 kDcollagenase). A direct and/or indirect inhibitor of an MMP may be used.

An optional component of the combination therapy described is a bisphosphonate. The bisphosphonates of the present invention correspond to the chemical formula: 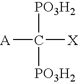
wherein A and X are independently selected from the group consisting of H, OH, halogen, NH₂, SH, phenyl, C1-C30 alkyl, C1-C30 substituted alkyl, C1-C10 alkyl or dialkyl substituted NH₂, C1-C10 alkoxy, C1-C10 alkyl or phenyl substituted thio, C1-C10 alkyl substituted phenyl, pyridyl, furanyl, pyrrolidinyl, imidazonyl, and benzyl.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

In the foregoing chemical formula, A can include X and X can include A such that the two moieties can form part of the same cyclic structure.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Preferred structures are those in which A is selected from the group consisting of H, OH, and halogen, and X is selected from the group consisting of C1-C30 alkyl, C1-C30 substituted alkyl, halogen, and C1-C10 alkyl or phenyl substituted thio.

More preferred structures are those in which A is selected from the group consisting of H, OH, and Cl, and X is selected from the group consisting of C1-C30 alkyl, C1-C30 substituted alkyl, Cl, and chlorophenylthio.

Most preferred is when A is OH and X is a 3-aminopropyl moiety, so that the resulting compound is a 4-amino-1,-hydroxybutylidene-1,1-bisphosphonate, i.e. alendronate.

Pharmaceutically acceptable salts and derivatives of the bisphosphonates are also useful herein. Nonlimiting examples of salts include those selected from the group consisting alkali metal, alkaline metal, ammonium, and mono-, di, tri-, or tetra-C1-C30-alkyl-substituted ammonium. Preferred salts are those selected from the group consisting of sodium, potassium, calcium, magnesium, and ammonium salts. Nonlimiting examples of derivatives include those selected from the group consisting of esters, hydrates, and amides.

“Pharmaceutically acceptable” as used herein means that the salts and derivatives of the bisphosphonates have the same general pharmacological properties as the free acid form from which they are derived and are acceptable from a toxicity viewpoint.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials.

Nonlimiting examples of bisphosphonates useful herein include the following:

Alendronic acid, 4-amino-1-hydroxybutylidene-1,1-bisphosphonic acid.

Alendronate (also known as alendronate sodium or monosodium trihydrate), 4-amino-1-hydroxybutylidene-1,1-bisphosphonic acid monosodium trihydrate.

Alendronic acid and alendronate are described in U.S. Pat. No. 4,922,007, to Kieczykowski et al., issued May 1, 1990, and U.S. Pat. No. 5,019,651, to Kieczykowski, issued May 28, 1991, both of which are incorporated by reference herein in their entirety.

Cycloheptylaminomethylene-1,1-bisphosphonic acid, YM 175, Yamanouchi (cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zolendronate).

The bisphosphonate may be administered in a dosage and manner found appropriate by a clinician such as those described for individual biphosphonates in the PDR. For example, when the bisphosphonate is pamidronate, the dose is 0.10-10 mg/kg, preferably 0.5-5 mg/kg.

In a further optional embodiment, inhibitors of pro-angiogenic growth factors are included in the combination. Such inhibitors can prevent the growth factor binding or inhibit its intracellular signaling.

In another embodiment, a steroidal anti-inflammatory agent is included in the combination.

In an optional embodiment, the composition of the invention can utilize a variety of steroidal anti-inflammatory agents, including but not limited to the following agents (including possible sources): dexamethasone (Merck), betamethasone (Schering), triamcinolone (Fujisawa), 6a-methylprednisolone (Duramed), prednisolone (Merck), prednisone (Roxane), hydrocortisone (Merck), cortisone (Merck), and fludrocortisone (Apothecon). Preferred agents are dexamethasone, betamethasone, triamcinolone, 6a-methylprednisolone, and prednisolone. Dexamethasone and prednisone are the most preferred steroidal anti-inflammatory agents.

The steroidal anti-inflammatory agent (SAID) may be administered in any manner found appropriate by a clinician in generally accepted efficacious dose ranges such as those described for individual SAIDs in the PDR.

As with the use of other chemotherapeutic drugs, the individual patient will be monitored in a manner deemed appropriate by the treating physician. Typically, no additional drug treatments will occur until, for example, the patient's neutrophil count is at least 1500 cells/mm³. Dosages can also be reduced if severe neutropenia or severe peripheral neuropathy occurs, or if a grade 2 or higher level of mucositis is observed, using the Common Toxicity Criteria of the National Cancer Institute.

The pharmaceutical compositions of this invention which are found in combination may be in the dosage form of solid, semi-solid, or liquid such as, e.g. suspension, aerosols, or the like. Preferably the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration. Compositions may be provided as sustained release or timed release formulations. The carrier or diluent may include any sustained release material known in the art, such as glyceryl monostrearate or glyceryl distearate, alone or mixed with a wax. Microencapsulation may also be used. The timed release formulation can provide a combination of immediate and pulsed release throughout the day. The diluent is selected so as not to affect the biological activity of the combination. Examples of such diluents are distilled water, physiological saline, Ringer's solution, dextrose solution, and Hank's solution. In addition, the pharmaceutical composition of formulation may also include other carriers, adjuvants, emulsifiers such as poloxamers, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

In therapeutic applications, the dosages and administration schedule of the agents used in accordance with the invention vary depending on the agent, the age, weight, and clinical condition of the recipient patient, and the experience and judgment of the clinician or practitioner administering the therapy, among other factors affecting the selected dosage. Generally, the dose and administration scheduled should be sufficient to result in slowing, and preferably regressing, the growth of the tumor(s) and also preferably causing complete regression of the cancer. In some cases, regression can be monitored by a decrease in blood levels of tumor specific markers. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped.

The agents in combination, or separately, are delivered at periodic intervals that can range from several times a day to once per month. As noted above, the agents are administered until the desired therapeutic outcome has been obtained. Additionally, in order to avoid side-effects not all components of the combination need to be delivered at each administration. For example, if the combination is administered twice a week the biphosphonates can be administered only once a week (every second treatment).

This invention further includes pharmaceutical combinations comprising a non-steroidal anti-inflammatory drug, a cytotoxic agent, an ester of benzoic acid, redox quinone and optionally, a bisphosphonate and/or MMP inhibitor and/or inhibitors of pro-angiogenic factors as provided above and kits for the treatment of cancer patients comprising a vial of the anti-inflammatory agent, a vial of the cytotoxic agent and a vial of the ester of benzoic acid, at the doses provided above. Preferably, the kits also include a vial of redox quinone. Most preferably, the kit contains instructions describing their use in combination.

In addition, the full treatment kit includes a formulation of at least two of the agents and/or a formulation of a cytotoxic agent alone and/or a formulation of benzyl benzoate and Vitamin K₃.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications cited herein are incorporated herein by reference.

### EXAMPLE 1

Experiment 1

In Vivo Testing

Breast cancer is a highly lethal disease. To test the efficacy of rofecoxib, benzyl benzoate, cyclophosphamide, and pamidronate alone and in combination, a mouse breast cancer cell line, EMT6, that was originally isolated from a spontaneous tumor in a BALB/c mouse was used (Twentyman P R and Watson J V, Br. J. Cancer 35: 120 (1977)). This line was further selected for a drug resistant variant, EMT6/CTX, that was used in this study (Teicher B A et al. Cancer Chemother. Pharmacol. 37: 601 (1996)). An EMT6/CTX cell suspension was freshly prepared in DMEM medium+10% FCS, following trypsinization of cell grown in tissue culture. Anesthesia is performed by injecting 0.08 ml per mouse of Ketamine 30 mg/ml+0.07% Chanazine in PBS.

Subcutaneous inoculation of 3×10⁵ cells at the shaved back of anesthetized C57BLXBALB/c F1 7-8 weeks old male mice results in palpable tumors within 4 days, and animals succumb to the tumor with around 4 weeks. Thus, this is a highly aggressive tumor growth model.

Drugs

Rofecoxib (Vioxx, a Merck & Co. Inc. product), as an oral suspension. Diclofenac, water-soluble Vitamin K₃ (menadione sodium bisulfite) and Benzyl benzoate, purchased from Sigma-Aldrich (St. Louise, Mich.), were formulated into solution in double-distilled water plus 2% Tween 20 (J.T. Baker, NJ). Cyclophosphamide, purchased from Bristol-Meyers Squibb (Princeton, N.J.), was formulated into solution in double-distilled water plus 2% Tween 20. Pamidronate (Pamidronic acid) was dissolved in 2M HEPES. The following doses were used for treatment in all three experiments: Rofecoxib 10 mg/kg, pamidronate 10 mg/kg, Diclofenac 30 or 60 mg/kg, menadione sodium bisulfite 20 or 50 mg/kg, cyclophosphamide (CTX) 50, 60 or 100 mg/kg and benzyl benzoate 80 or 550 mg/kg. Vehicle: 2% Tween 20 in double distilled water. The preparations were made in a concentration that will require an i.p. injection of 0.1 ml per 20 gr body weight to achieve the above doses.

Design of Animal Trials

Six mice per group; each treatment per mouse, twice a week:
Group 1. Control vehicle;
Group 2. Benzyl benzoate 550 mg/kg (A) and Pamidronate 10 mg/kg (B)
Group 3. B and Rofecoxib 10 mg/kg (C)
Group 4. A and C

All treatments were started 5 days after cell inoculation. Treatment comprised an intraperitoneal injection of 0.1 ml per 20 gr body weight of the formulation. Mice were treated twice a week for a period of 3 weeks.

Tumor dimensions were measured twice a week. Tumor volume was calculated according to conventional methods (volume=a²×b×0.52 where a represents the tumor width and b is its length) and the data are represented as changes in tumor volume with time. Toxicity was evaluated by qualitative observation of the general appearance and behavior of the mice, by measuring their body weight during various intervals during the course of the treatments and by obtaining mortality data.

The results of this experiment are set forth in FIG. 1. As can be seen, none of the combination of two agents has a significant effect on the tumor volume.

Experiment 2

The tumor model, drug formulation and tumor volume measurements were identical to Experiment 1. Again, 6 mice were used for each group. This experiment tested the effect of the drug combination of A+B+C.

Treatment per mouse twice a week:
Group 1. Control vehicle;
Group 2. ABC

The treatment started 5 days after cell inoculation. Treatment comprised an intraperitoneal injection of 0.1 ml per 20 gr body weight of the formulation. Mice were treated twice a week for a period of 3 weeks.

The results of this experiment are set forth in FIG. 2. As can be seen, the combination ABC dramatically reduced tumor growth. Mice exhibited less than 10% weight loss and no toxicity was observed.

Experiment 3

The tumor model, drug formulation and tumor volume measurements were identical to Experiment 1. Seven mice were used for each group. This experiment tested the effect of cyclophosphamide by itself and with the drug combination of ABC.
Group 1. Control vehicle
Group 2. Cyclophosphamide 50 mg/kg (CTX 50)
Group 3. ABC
Group 4. ABC+CTX50

The treatment started 3 days after cell inoculation. Treatment comprised an intraperitoneal injection of 0.1 ml per 20 gr body weight of the formulation. Mice were treated twice a week for a period of 4 weeks.

The results are presented in FIG. 3 and demonstrated the superiority of the X4 combination protocol. While CTX 50 and ABC each by itself has a moderate effect on tumor growth, the X4 combination of ABC+CTX 50 yielded a synergistic effect.

Experiment 4

The tumor model, drug formulation and tumor volume measurements were identical to Experiment 1. Seven mice were used for each group. This experiment tested the effect of cyclophosphamide by itself, with the drug combination of ABC and the effect of administrating small doses of A (1/6) 6 times a week.
Group 1. Control vehicle
Group 2. Cyclophosphamide 50 mg/kg (CTX 50)
Group 3. CTX 50+ABC
Group 4. (CTX 50+BC)×2 per week+1/6A×6 per week (=A all week)

The treatment started 4 days after cell inoculation. Treatment comprised an intraperitoneal injection of 0.1 ml per 20 gr body weight of the formulation. Mice were treated twice a week (group 4 was treated 6 times a week) for a period of 4 weeks.

The results are presented in FIG. 4 and demonstrated the superiority of spreading the dose of A throughout the week.

Experiment 5

The tumor model, drug formulation and tumor volume measurements were identical to Experiment 1. Seven mice were used for each group. This experiment tested the effect of changing the treatment regime: once a week vs. twice and injecting BC together with CTX on the same day vs. on different days of the week
Group 1. Control vehicle
Group 2. (BC+CTX 50)×2 per week; A all week (Sun.-Fri.)
Group 3. (BC+CTX 100) once a week; A all week (Sun.-Fri.)
Group 4. BC Wed.; CTX 100 Sun.; A all week (Sun.-Fri.)

The treatment started 4 days after cell inoculation. Treatment comprised an intraperitoneal injection of 0.1 ml per 20 gr body weight of the formulation. Mice were treated once, twice or 6 times a week for a period of 4 weeks, according to the above protocol.

The results are presented in FIG. 5 and demonstrated the possible advantage of injecting BC and CTX on different days of the weekly cycle.

Experiment 6

The tumor model, drug formulation and tumor volume measurements were identical to Experiment 1. Seven mice were used for each group. This experiment tested the replacement of B by doubling the dose of C (2C no B) and the usage of Diclofenac 25 mg/kg (D) with B or twice its dose (2D) without B, as the NSAID of choice.
Group 1. Control vehicle
Group 2. BC Wed.; CTX 100 Sun.; A all week
Group 3. 2C Wed.; CTX 100 Sun.; A all week
Group 4. BD Wed.; CTX 100 Sun.; A all week
Group 5. 2D Wed.; CTX 100 Sun.; A all week

The treatment started 3 days after cell inoculation. Treatment comprised an intraperitoneal injection of 0.1 ml per 20 gr body weight of the formulation. Mice were treated 6 times a week for a period of 4 weeks.

The results are presented in FIG. 6 and demonstrated that larger doses (×2) of NSAIDs can be used to substitute the need of B. FIG. 6 also demonstrated the superiority of D (25 and 50 mg/kg) over C (5 and 10 mg/kg, respectively).

Experiment 7

The tumor model, drug formulation and tumor volume measurements were identical to Experiment 1. Seven mice were used for each group. This experiment tested the effect of changing the time intervals between administration of CTX and D (1, 3 or 5 days apart) and the “spreading” of the CTX dose throughout the week (20 mg/kg 6 times a week).
Group 1. Control vehicle
Group 2. CTX 100 Sun.; D50 Wed.; A all week
Group 3. CTX 100 Sun.; D50 Mon.; A all week
Group 4. CTX 100 Sun.; D50 Fri.; A all week
Group 5. CTX20 all week (Sun-Fri); D50 Wed.; A all week

The treatment started 5 days after cell inoculation. Treatment comprised an intraperitoneal injection of 0.1 ml per 20 gr body weight of the formulation. Mice were treated 6 times a week for a period of 4 weeks.

The results are presented in FIG. 7 and demonstrated that treatment with CTX 100 once a week is preferred over equal small doses throughout the week and that the interval between administration of CTX and D should be at most 1 day.

Experiment 8

The tumor model, drug formulation and tumor volume measurements were identical to Experiment 1. Seven mice were used for each group. This experiment tested the effect of the addition of Vitamin K₃ (VK) at doses of 20 mg/kg 6 times a week or 50 mg/kg twice a week, to the formulation.

The days of the week are abbreviated as follows: Sun. (S), Mon. (M), Tues. (T), Wed. (W), Thu. (H), and Fri (F).

Group 1: Vehicle only (Control)
Group 2: (A+VK20) all week
Group 3: (CTX60+D30+VK50)M,H+(A) all week
Group 4: (CTX60+D30)M,H+(A+VK20) all week

The treatment started 4 days after cell inoculation. Treatment comprised an intraperitoneal injection of 0.1 ml per 20 gr body weight of the formulation. Mice were treated 6 times a week for a period of 4 weeks.

The results are presented in FIG. 8 and demonstrated that (a) the addition of VK improve the efficacy of the treatment; (b) the administration of VK at lower doses throughout the week (every day) is preferable.

### EXAMPLE 2

Description of the Drug Product

The treatment was a combination of four drugs supplied for a specific dosing schedule. The drugs included Cyclophosphamide (CTX), Diclofenac (Voltaren), vitamin K3 (Menadione) and Benzyl Benzoate that was provided as part of a suspension suitable for oral administration. All these drugs were formulated in an oral suspension composed of 2% Lutrol® F68 and 2% Solutol® HS-15 (suspending agents) and 1.15% Benzyl Benzoate, in water. The drug combination was prepared under sterile, GMP conditions in the Sheba Medical center pharmacy. The drug combination was supplied in 25 ml, 37.5, and 50 ml doses. The amounts of the drugs supplied were as follows:

25 ml Dose for Use Twice a Week:

| | |
|---|---|
| Cyclophosphamide | 200 mg (approximately 60 mg/kg/week)* |
| Diclofenac | 100 mg (approximately 30 mg/kg/week)* |
| Vitamin K3 | 70 mg (approximately 7 mg/kg/week)* |

25 ml Dose for Use on the Rest of the Week (5 days):

| | |
|---|---|
| Vitamin K3 | 70 mg |

37.5 ml Dose for Use Twice a Week:

| | |
|---|---|
| Cyclophosphamide | 300 mg (approximately 90 mg/kg/week)* |
| Diclofenac | 150 mg (approximately 45 mg/kg/week)* |
| Vitamin K3 | 105 mg (approximately 10.5 mg/kg/week)* |

37.5 ml Dose for Use on the Rest of the Week (5 days):

| | |
|---|---|
| Vitamin K3 | 105 mg |

50 ml Dose for Use Twice a Week:

| | |
|---|---|
| Cyclophosphamide | 400 mg (approximately 120 mg/kg/week)* |
| Diclofenac | 200 mg (approximately 60 mg/kg/week)* |
| Vitamin K3 | 140 mg (approximately 14 mg/kg/week)* |

*assuming an average body weight of 70 kg

50 ml Dose for Use on the Rest of the Week (5 days):

| | |
|---|---|
| Vitamin K3 | 140 mg |

The drug combination was supplied in numbered tubes (1-7), one for each day of the week. The tubes were shaken well before use, to insure homogenization of the suspension.

The tubes were color-coded as follows:

Tubes with blue caps contain the cytotoxic agent and were administered twice weekly (days #1 and #4). Gloves were worn when using these tubes and they were handled with extra care. Tubes with red caps were taken on the other five days of the week (2, 3, 5, 6, and 7). All tubes were stored in a freezer (−20° C.) until distributed to patients. The patients kept the tubes stored in a refrigerator, protected from light and out of the reach of children.

Initial Dose and Scheduling

The suspension was taken orally in the evening during or after dinner. The drug was supplied so that the patient received Vitamin K3 daily. Cyclophosphamide and Diclofenac were administered twice weekly in combination with Vitamin K3. The initial dose was 25 ml administered daily for two weeks. If no significant toxicity occurred, the next dose of 37.5 ml was administered for two weeks. If the patient tolerated this dose, treatment was continued with administration of 50 ml of drug combination for eight weeks.

Overall Design of the Clinical Trial

2 wks 50% dose
2 wks 75% dose
8 wks 100% dose
Imaging at 0, 6 and 12 wks
   Safety Profile

No major side effects were observed during 1 to 3 months treatment of 30 patients (except for a manageable bitter taste).

Preliminary Efficacy

Early signs of stable disease or regression in 7 out of 12 patients were evaluated at 12 wks (imaging).

2 colon cancer patients were stable after 20 wks on combinations (vs. a steady progression beforehand despite treatment).

In one patient an MRI taken before the initiation of the drug-combination treatment showed a tumor mass that compressed the right urethra and interfered with urine flow.

An MRI of the same section kidney was taken two months after the initiation of the drug-combination treatment showed a significant shrinkage of the tumor and an improvement of the kidney condition.

Although the foregoing invention has been described in some detail by way of illustration and example for the purposes of clarity of understanding, one skilled in the art will easily ascertain that certain changes and modifications may be practiced without departing from the spirit and scope of the appended claims.

1. A pharmaceutical composition comprising a cytotoxic agent, a non-steroidal anti-inflammatory drug (NSAID), an ester of benzoic acid and a pharmaceutically acceptable carrier.

2. The pharmaceutical composition of claim 1, further comprising a redox quinone.

3. The pharmaceutical composition of claims 1 or 2, further comprising an inhibitor of MMP.

4. The pharmaceutical composition of claims 1 or 2, further comprising a bisphosphonate.

5. The pharmaceutical composition of claim 4, wherein the bisphosphonate is selected from the group consisting of etidronate, pamidronate, clodronate, alendronate, tiludronate, ibandronate and risedronate.

6. The pharmaceutical composition of claim 5, wherein the bisphosphonate is pamidronate and alendronate.

7. The pharmaceutical composition of claims 1 or 2, wherein the NSAID is a COX1-2 inhibitor.

8. The pharmaceutical composition of claim 7, wherein the COX1-2 inhibitor is diclofenac or indomethacin.

9. The pharmaceutical composition of claims 1 or 2, wherein the ester of benzoic acid is benzyl benzoate.

10. The pharmaceutical composition of claims 1 or 2, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

11. The pharmaceutical composition of claims 1 or 2, wherein the cytotoxic agent is cyclophosphamide or ifosfamide.

12. The pharmaceutical composition of claims 1 or 2, further comprising a steroidal anti-inflammatory agent.

13. The pharmaceutical composition of claim 12, wherein the steroidal anti-inflammatory agent is dexamethasone or prednisone.

14. The pharmaceutical composition of claims 1 or 2, wherein the cytotoxic agent, NSAID, redox quinone, an ester of benzoic acid and the pharmaceutically acceptable carrier are formulated as sustained-release formulations.

15. The pharmaceutical composition of claims 1 or 2, further comprising inhibitors of pro-angiogenic growth factors.

16. The pharmaceutical composition of claim 2, wherein the redox quinone is Vitamin K₃.

17. A method of treating cancer comprising administering to a host in need of treatment an effective amount of a cytotoxic agent, a NSAID, an ester of benzoic acid and a pharmaceutically acceptable carrier.

18. The method of claim 17, further comprising administering a redox quinone.

19. The method of claim 18, wherein the redox quinone is formulated with benzyl benzoate.

20. The method of claims 17 or 18, further comprising administering an inhibitor of MMP.

21. The method of claims 17 or 18, further comprising administering a bisphosphonate.

22. The method of claims 17 or 18, further comprising administering inhibitors of pro-angiogenic growth factors.

23. The method of claims 17 or 18, wherein the cancer is a solid tumor or leukemia.

24. The method of claim 23, wherein the solid tumor is selected from the group consisting of lung cancer, colorectal cancer, breast cancer, prostate cancer, a brain tumor and melanoma.

25. A method of treating macular degeneration comprising administering to a host in need of treatment an effective amount of a cytotoxic agent, a NSAID, an ester of benzoic acid, redox quinone and a pharmaceutically acceptable carrier.

26. A method of treating obesity comprising administering to a host in need of treatment an effective amount of a cytotoxic agent, a NSAID, an ester of benzoic acid, redox quinone and a pharmaceutically acceptable carrier.

27. The method of claim 20, wherein the MMP inhibitor is doxycycline or CMT-8.

28. The method of claim 21, wherein the bisphosphonate is selected from the group consisting of etidronate, pamidronate, clodronate, alendronate, tiludronate, ibandronate and risedronate.

29. The method of claim 28, wherein the bisphosphonate is pamidronate and alendronate.

30. The method of claims 17, 18, 25 or 26, wherein the NSAID is a COX1-2 inhibitor.

31. The method of claim 30, wherein the COX1-2 inhibitor is diclofenac or indomethacin.

32. The method of claims 17, 18, 25 or 26, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

33. The method of claim 32, wherein the cytotoxic agent is cyclophosphamide or ifosfamide.

34. The method of claims 17, 18, 25 or 26, wherein, the aromatic ester of benzoic acid is benzyl benzoate.

35. The method of claims 17, 18, 25 or 26, further comprising administering a steroidal anti-inflammatory agent.

36. The method of 35, wherein the steroidal anti-inflammatory agent is dexamethasone or prednisone.

37. The method of 18, wherein the wherein the redox quinone is Vitamin K₃.

38. The method of claims 17, 18, 25 or 26, wherein the composition is formulated as a controlled release.

39. The method of claims 17, 18, 25 or 26, wherein the host is selected from the group consisting of a human, cat, dog or horse.

40. A kit for the treatment of cancer, macular degeneration or obesity comprising separate vials containing a cytotoxic agent, NSAID, an ester of benzoic acid and a pharmaceutically acceptable carrier and directions for administration of each component.

41. The kit of claim 40, further comprising a vial containing a redox quinone.

42. The kit of claims 40 or 41, further comprising a vial containing an MMP inhibitor.

43. The kit of claims 40 or 41, further comprising a vial containing a bisphosphonate.

44. The kit of claim 43, wherein the bisphosphonate is selected from the group consisting of etidronate, pamidronate, clodronate, alendronate, tiludronate, ibandronate and risedronate.

45. The kit of claim 44, wherein the bisphosphonate is pamidronate and alendronate.

46. The kit of claims 40 or 41, wherein the NSAID is a COX1-2 inhibitor.

47. The kit of 46, wherein the COX1-2 inhibitor is diclofenac or indomethacin.

48. The kit of claims 40 or 41, wherein the ester of benzoic acid is benzyl benzoate.

49. The kit of claim 41, wherein the redox quinone is formulated with benzyl benzoate.

50. The kit of claim 41, wherein the redox quinone is Vitamin K₃.

51. The kit of claims 40 or 41, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

52. The kit of claim 51, wherein the cytotoxic agent is cyclophosphamide or ifosfamide.

53. The kit of claims 40 or 41, further comprising a steroidal anti-inflammatory agent.

54. The kit of claim 53, wherein the steroidal anti-inflammatory agent is dexamethasone or prednisone.

55. The kit of claim 42, wherein the MMP inhibitor is doxycycline or CMT-8.

56. The kit of claims 40 or 41 further comprising an inhibitor of pro-angiogenic growth factor is an agent.

57. A method of treating cancer, macular degeneration or obesity in a host in need thereof, comprising administering one or a combination of agents daily for at least one week, wherein the agents are selected from a cytotoxic agent, an NSAID, a redox quinone and an ester of benzoic acid, wherein the combination of agents is administered such that over at least a two day period the same combination of agents are not administered, and optionally for at least one day of the week a placebo is administered to the host.

58. The method of claim 57, wherein an inhibitor of pro-angiogenic growth factor is an agent.

59. The method of 57, wherein an inhibitor of an MMP is an agent.

60. The method of claim 57, wherein a bisphosphonate is an agent.

61. The method of claim 59, wherein the MMP inhibitor is doxycycline and CMT-8.

62. The method of claim 60, wherein the bisphosphonate is selected from the group consisting of etidronate, pamidronate, clodronate, alendronate, tiludronate, ibandronate and risedronate.

63. The method of claim 57, wherein the NSAID is a COX1-2 inhibitor.

64. The method of claim 63, wherein the COX1-2 inhibitor is diclofenac or indomethacin.

65. The method of claim 57, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

66. The method of claim 65, wherein the cytotoxic agent is cyclophosphamide or ifosfamide.

67. The method of claim 57, wherein the ester of benzoic acid is benzyl benzoate.

68. The method of claim 57, wherein the redox quinone is Vitamin K₃.

69. The method of claim 68, wherein Vitamin K₃ is formulated with benzyl benzoate.

70. The method of claim 57, wherein a steroidal anti-inflammatory agent is an agent.

71. The method of claim 70, wherein the steroidal anti-inflammatory agent is dexamethasone or prednisone.

72. The method of claim 57, wherein the host is selected from the group consisting of a human, cat, dog or horse.

73. A pharmaceutical composition comprising a cytotoxic agent, an NSAID, a redox quinone, a benzyl benzoate and a pharmaceutically acceptable carrier.

74. The pharmaceutical composition of claim 73, wherein the composition is formulated for oral administration.

75. The pharmaceutical composition of claim 73, wherein the NSAID is a COX1-2 inhibitor.

76. The pharmaceutical composition of claim 75, wherein the COX1-2 inhibitor is diclofenac or indomethacin.

77. The pharmaceutical composition of claim 73, wherein the cytotoxic agent is selected from the group consisting of cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, and paclitaxel.

78. The pharmaceutical composition of claim 73, wherein the redox quinone is Vitamin K₃.

79. A pharmaceutical composition for oral administration comprising cyclophosphamide, diclofenac, Vitamin K₃, benzyl benzoate and a pharmaceutically acceptable carrier.
